(19) United States
(12) Patent Application Publication
(10) Pub. No.: US 2010/0107281 A1
(43) Pub. Date: Apr. 29, 2010

(54) PESTICIDAL COMBINATIONS

(75) Inventor: Alex Cochran, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/444,924

(22) PCT Filed: Oct. 4, 2007
(86) PCT No.: PCT/EP07/08599

§ 371 Date: Apr. 9, 2009

Related U.S. Application Data
(60) Provisional application No. 60/828,680, filed on Oct. 9, 2006.
(60) Provisional application No. 60/894,775, filed on Mar. 14, 2007.

(51) Int. Cl. A01H 5/00; A01N 43/828; A01P 5/00; A01N 25/26

(52) U.S. Cl. 800/298; 514/361; 504/100

(57) ABSTRACT

A method of controlling or preventing nematode and/or pathogenic damage in a plant propagation material, a plant, and/or plant organs that grow at a later point in time, which comprises applying on the plant, part of the plant, or the surrounding area thereof, a pesticidal combination comprising, for example, at least two active ingredient components optionally together with one or more customary formulation auxiliaries, wherein component (I) is one or more nematicide(s) and component (II) is one or more plant activator(s), in any desired sequence or simultaneously. Furthermore, the invention relates to a method of controlling or preventing nematode and/or pathogenic damage in a plant propagation material, a plant, and/or plant organs that grow at a later point in time, which comprises applying on the plant, part of the plant, or the surrounding area thereof, a pesticidal combination comprising two or more nematicides optionally together with one or more customary formulation auxiliaries, in any desired sequence or simultaneously.

The present invention relates to the use of a defined combination of pesticidal active ingredients, and compositions thereof, and methods for using such combinations in the control or prevention of nematode and/or pathogenic damage, in particular in a plant propagation material and plant organs that grow at a later point in time by applying the combination on to the plant propagation material.

Nematodes damage crops by direct feeding damage, by transmitting viruses and by facilitating bacterial and fungal infections. The damage caused by nematodes to crops is often unspecific and easily confused with drought, malnutrition or disease. Typical symptoms are wilting, yellowing of the foliage and uneven or stunted growth.

Methods to control nematodes and thereby protect the plant include (1) use of nematicides (e.g., aldicarb) and fumigants (e.g., methyl bromide), (2) use of soil steaming, (3) use of crop rotation practices, which is effective against nematodes that are specific to a particular crop; however, nematodes that have different hosts cannot be controlled by this method, and (4) use of nematode resistant or tolerant crops, which have been developed by conventional breeding or recombinant DNA technology (genetically modified plants).

Certain combinations of active ingredients for controlling nematodes and/or pathogenic damage are described in the literature. The biological properties of those known combinations are not entirely satisfactory in the areas of pathogenic control, phytotoxicity, and environmental and worker exposure, for example. In particular, in the instance a pathogen has become, or risks becoming resistant to the previously known combinations, improved methods of control or prevention are sought.

There is a continuing need to provide pesticidal combinations, which provide improved, for example, biological properties, for example, synergistic properties, especially for controlling nematodes and/or pathogenic damage.

That need is solved according to the invention by the provision of the present pesticidal combination. Accordingly, in a first aspect, the present invention provides a pesticidal combination comprising at least two active ingredient components optionally together with one or more customary formulation auxiliaries, wherein component (I) is one or more nematicide(s) and component (II) is one or more plant activator(s).

The term “hematicide” refers to a compound having an effect on, such as reduction in the damage caused by, agricultural-related nematodes. Non-limiting examples of nematicides include avermectins (e.g., abamectin), carbamate nematicides (e.g., alanycarb, aldicarb, thiodicarb, carbofuran, carbosulfan, oxamyl), organophosphorus nematicides (e.g., cadusafos, chlorpyrifos, dimethoate, ethoprophos, fenamiphos, fosthiazate, heterophos, phorate, phosphamidon, phosphocarb, terbufos, triazophos), fumigants (e.g., chloropicrin, dazomet, DCIP, metam, methyl bromide, methyl iodide, methyl isothiocyanate) and certain fungicides (e.g., benomyl, captan, thiabendazole, thiophanate-methyl). Particularly preferred nematicides include abamectin, thiodicarb, aldicarb benomyl, captan, oxamyl, thiabendazole and thiophanate-methyl. More particularly preferred nematicides include abamectin, thiodicarb, aldicarb and oxamyl.

The term “plant activator” refers to a compound which does not act directly on the disease organism, nor does it alter the DNA of treated plants, but instead activates a natural defense mechanism in the host plant, referred to as systemic acquired resistance (SAR). Non-limiting examples of plant activators include acibenzolar-S-methyl, CGA 210007, benzoic acid, harpin, magnesium-dihydrojasmonate and salicylic acid. Particularly preferred plant activators include acibenzolar-S-methyl and harpin.

Another embodiment of the invention relates to a pesticidal combination comprising two or more nematicides optionally together with one or more customary formulation auxiliaries.

Each of the combinations demonstrates unexpected, for example synergistic, activity compared to the activity of components alone.

In a second aspect, the present invention provides a method of controlling or preventing nematode and/or pathogenic damage in a plant propagation material, a plant, and/or plant organs that grow at a later point in time, which comprises applying to the plant, part of the plant, or the surrounding area thereof the combination, as defined in the first aspect, in any desired sequence or simultaneously.

In a third aspect, the present invention provides a method of protecting a plant propagation material, a plant, and/or plant organs that grow at a later point in time against nematode and/or pathogenic damage, which comprises applying to the plant, part of the plant, or the surrounding area thereof the combination, as defined in the first aspect, in any desired sequence or simultaneously.

In a fourth aspect, the present invention provides a method which comprises (i) treating a plant propagation material, such as a seed, with a pesticidal combination as defined in the first aspect, and (ii) planting or sowing the treated propagation material, wherein the combination protects against nematode and/or pathogenic damage of the treated plant propagation material, parts of plant and/or plant grown from the treated propagation material.

In a fifth aspect, the present invention provides a method which comprises (i) treating a plant propagation material, such as a seed, with a pesticidal combination as defined in the first aspect, and (ii) planting or sowing the treated propagation material, and (iii) achieving protection against nematode and/or pathogenic damage of the treated plant propagation material, parts of plant and/or plant grown from the treated propagation material.

In a sixth aspect, the present invention provides a method as defined above, wherein the active ingredient components of the combination, as defined in first aspect, are applied simultaneously.

In a seventh aspect, the present invention provides a method as defined above, wherein the combination, as defined in the first aspect, is applied on plant propagation material.

In an eighth aspect, the present invention relates to a plant propagation material treated with the combination as defined in the first aspect.

The components (I) and (II) defined in the first aspect are active ingredients for use in the agrochemical industry (also known as pesticides). A description of their structure as well as other pesticides (e.g., fungicides, insecticides) can be found in The e-Pesticide Manual, Version 3.2, 13^{th} Edition, Editor C. D. S. Tomlin, British Crop Protection Council, 2005-06, with the exception of heterophos, phosphocarb, thiophanate-methyl, benzoic acid, salicylic acid, harpin and magnesium-dihydrojasmonate for which alternative references have been provided.

CGA 210007 (CAS RN 35272-27-6) was disclosed as a plant activator, for example, in European Journal of Plant Pathology (2002), 108(1), 41-49.

Heterophos (CAS RN 40626-35-5) was disclosed as an organophosphorus nematicide, for example, in U.S. Pat. No. 4,242,333.

Phosphocarb (CAS RN 126069-54-3) was disclosed as an organophosphorus nematicide, for example, in U.S. Pat. No. 4,855,140.

Thiophanate-methyl (CAS RN 23564-05-8) was disclosed as a nematicide, for example, in International Journal of Biology and Biotechnology (2004), 1(4), 613-618.

Benzoic acid (CAS RN 65-85-0) and salicylic acid (CAS RN 69-72-7) were disclosed as plant activators, for example, in EP 1,036,499.

Harpin (CAS RN 151438-54-9) was disclosed as a plant activator, for example, in WO 95/31564.

Magnesium-dihydrojasmonate was disclosed as a plant activator, for example, in Agrow Magazine (2006), 5, 23.

Controlling, preventing or protecting and its inflections, within the context of the present invention, mean reducing any undesired effect, such as

nematode damage on, and
pathogenic, such as phytopathogenic, especially fungi, infestation or attack of,
a plant, part of the plant or plant propagation material to such a level that an improvement is demonstrated.

The pesticidal combinations according to the invention have very advantageous properties for protecting plants against (i) nematode attack or damage and/or (ii) pathogenic, such as phytopathogenic, especially fungi, attack or infestation, which result in a disease and damage to the plant; particularly in instance of plants, the present invention can control or prevent nematode and/or pathogenic damage on a seed, parts of plant and/or plant grown from the treated seed. In some cases, control against nematode attack or damage also indirectly results in control against pathogenic attack, and vice-a-versa.

These properties are, for example, the synergistically enhanced action of combinations of components (I) and (II), resulting in lower nematode and/or pathogenic damage, lower rates of application, or a longer duration of action. In the instance of agriculture, the enhanced action is found to show an improvement in the growing characteristics of a plant by, for example, higher than expected control of nematode and/or pathogenic damage.

The improvement in the growing (or growth) characteristics of a plant can manifest in a number of different ways, but ultimately it results in a better product of the plant. It can, for example, manifest in improving the yield and/or vigour of the plant or quality of the harvested product from the plant, which improvement may not be not connected to the control of nematodes.

As used herein the phrase “improving the yield” of a plant relates to an increase in the yield of a product of the plant by a measurable amount over the yield of the same product of the plant produced under the same conditions, but without the application of the subject method. It is preferred that the yield be increased by at least about 0.5%, more preferred that the increase be at least about 1%, even more preferred is about 2%, and yet more preferred is about 4%, or more. Yield can be expressed in terms of an amount by weight or volume of a product of the plant on some basis. The basis can be expressed in terms of time, growing area, weight of plants produced, amount of a raw material used, or the like.

As used herein the phrase “improving the vigour” of a plant relates to an increase or improvement of the vigour rating, or the stand (the number of plants per unit of area), or the plant height, or the plant canopy, or the visual appearance (such as greener leaf colour), or the root rating, or emergence, or protein content, or increased tillering, or bigger leaf blade, or less dead basal leaves, or stronger tillers, or less fertilizer needed, or less seeds needed, or more productive tillers, or earlier flowering, or early grain maturity, or less plant verse (lodging), or increased shoot growth, or earlier germination, or any combination of these factors, or any other advantages familiar to a person skilled in the art, by a measurable or noticeable amount over the same factor of the plant produced under the same conditions, but without the application of the subject method. When it is said that the present method is capable of “improving the yield and/or vigour” of a plant, the present method results in an increase in either the yield, as described above, or the vigour of the plant, as described above, or both the yield and the vigour of the plant.

Accordingly, the present invention also provides a method of improving the growing characteristics of a plant, and/or plant organs that grow at a later point in time, which comprises applying to the plant, part of the plant, or the surrounding area thereof the combination, as defined in the first aspect, in any desired sequence or simultaneously.

In a preferred embodiment of any aspect of the invention, each combination is a composition comprising, preferably of, components (I) and (II), and optionally one or more customary formulation auxiliaries.

In a preferred embodiment of any aspect of the invention, are combinations of the invention wherein component (I) is one nematicide.

In another preferred embodiment of any aspect of the invention, are combinations of the invention wherein component (I) is a mixture of two nematicides.

In a preferred embodiment of any aspect of the invention, are combinations of the invention wherein component (II) is one plant activator.

In a preferred embodiment of any aspect of the invention, are combinations of the invention wherein component (I) is one nematicide and component (II) is one plant activator.

In another preferred embodiment of any aspect of the invention, are combinations of the invention wherein component (I) is a mixture of two nematicides and component (II) is one plant activator.

Preferred embodiments are combinations comprising

(a) abamectin as component (I) and acibenzolar-S-methyl as component (II);
(b) aldicarb as component (I) and acibenzolar-S-methyl as component (II);
(c) oxamyl as component (I) and acibenzolar-S-methyl as component (II); and
(d) thiodicarb as component (I) and acibenzolar-S-methyl as component (II).

Further preferred embodiments are combinations comprising

(e) a mixture of abamectin and benomyl as component (I) and acibenzolar-S-methyl as component (II);
(f) a mixture of abamectin and captan as component (I) and acibenzolar-S-methyl as component (II);
(g) a mixture of abamectin and thiabendazole as component (I) and acibenzolar-S-methyl as component (II);
(h) a mixture of abamectin and thiophanate-methyl as component (I) and acibenzolar-S-methyl as component (II);
(j) a mixture of aldicarb and benomyl as component (I) and acibenzolar-S-methyl as component (II);
(k) a mixture of aldicarb and captan as component (I) and acibenzolar-S-methyl as component (II);
(m) a mixture of aldicarb and thiabendazole as component (I) and acibenzolar-S-methyl as component (II);
(n) a mixture of aldicarb and thiophanate-methyl as component (I) and acibenzolar-S-methyl as component (II);
(o) a mixture of oxamyl and benomyl as component (I) and acibenzolar-S-methyl as component (II);
(p) a mixture of oxamyl and captan as component (I) and acibenzolar-S-methyl as component (II);
(q) a mixture of oxamyl and thiabendazole as component (I) and acibenzolar-S-methyl as component (II);
(r) a mixture of oxamyl and thiophanate-methyl as component (I) and acibenzolar-S-methyl as component (II);
(s) a mixture of thiodicarb and benomyl as component (I) and acibenzolar-S-methyl as component (II);
(t) a mixture of thiodicarb and captan as component (I) and acibenzolar-S-methyl as component (II);
(u) a mixture of thiodicarb and thiabendazole as component (I) and acibenzolar-S-methyl as component (II); and
(v) a mixture of thiodicarb and thiophanate-methyl as component (I) and acibenzolar-S-methyl as component (II).

Other preferred embodiments are combinations comprising

(a′) benomyl as component (I) and acibenzolar-S-methyl as component (II);
(b′) captan as component (I) and acibenzolar-S-methyl as component (II);
(c′) thiabendazole as component (I) and acibenzolar-S-methyl as component (II); and
(d′) thiophanate-methyl as component (I) and acibenzolar-S-methyl as component (II).

Other preferred embodiments are combinations comprising

(e′) a mixture of abamectin and benomyl;
(f′) a mixture of abamectin and captan;
(g′) a mixture of abamectin and thiabendazole;
(h′) a mixture of abamectin and thiophanate-methyl;
(j′) a mixture of aldicarb and benomyl;
(k′) a mixture of aldicarb and captan;
(m′) a mixture of aldicarb and thiabendazole;
(n′) a mixture of aldicarb and thiophanate-methyl;
(o′) a mixture of oxamyl and benomyl;
(p′) a mixture of oxamyl and captan;
(q′) a mixture of oxamyl and thiabendazole;
(r′) a mixture of oxamyl and thiophanate-methyl;
(s′) a mixture of thiodicarb and benomyl;
(t′) a mixture of thiodicarb and captan;
(u′) a mixture of thiodicarb and thiabendazole; and
(v′) a mixture of thiodicarb and thiophanate-methyl.

Each of the combinations of the invention can be used in the agricultural sector and related fields of use for controlling or preventing nematode and/or pathogenic damage on plants.

Each of the combinations according to the present invention is effective against nematodes that can be inhibited using such a treatment regimen. These include, but are not limited to, root-knot nematodes, cyst-forming nematodes, stem eelworms and foliar nematodes. In particular, nematodes of the following species can be managed using the combinations of the invention: Anguina spp., Aphelenchoides spp., Belonolaimus spp., Criconemella spp., Criconemoides spp., Ditylenchus spp., Dolichodorus spp., Globodera spp. (e.g., Globodera rostochiensis), Helicotylenchus spp., Heterodera spp. (e.g., Heterodera schachtii, Heterodora avenae, Heterodera glycines, and Heterodora trifolii), Hemicriconemoides spp., Hemicycliophora spp., Hirschmaniella spp., Hoplolaimus spp., Hypsoperine spp., Longidorus spp., Macroposthonia spp., Melinius spp., Meloidogyne spp. (e.g., Meloidogyne incognita and Meloidogyne javanica), Nacobbus spp., Paratrichodorus spp., Pratylenchus spp. (e.g., Pratylenchus neglectans and Pratylenchus penetrans), Punctodera spp., Quinisulcius spp., Radopholus spp. (e.g., Radopholus similis), Rotylenchulus spp., Scutellonema spp., Subanguina spp., Trichodorus spp., Tylenchorhynchus spp., Tylenchulus spp. (e.g., Tylenchulus semipenetrans) and Xiphinema spp.

The combinations of the present invention are particularly effective against root-knot nematodes of the species Meloidogyne spp. (e.g., Meloidogyne incognita) and cyst-forming nematodes of the species Heterodera spp. (e.g., Heterodera glycines “soybean cyst nematode”).

Each of the combinations according to the present invention is effective against phytopathogenic fungi, especially those occurring in plants, including seed-borne fungi. In particular, fungi of the following classes can be managed using the combinations of the invention: Ascomycetes (e.g., Penicillium, Gaeumannomyces graminis); Basidiomycetes (e.g., the genus Hemileia, Rhizoctonia, Puccinia); Fungi imperfecti (e.g., Botrytis, Helminthosporium, Rhynchosporium, Fusarium, Septoria, Cercospora, Alternaria, Pyricularia, Thelaviopsis and Pseudocercosporella herpotrichoides); Oomycetes (e.g., Phytophthora, Peronospora, Bremia, Pythium, Plasmopara and Aphanomyces); Zygomycetes (e.g., Rhizopus spp.). A combination is especially effective against Alternaria spp., Aspergillus spp., Claviceps purpurea, Cochliobolus spp., Colletotrichum spp., Diplodia maydis, Erysiphe graminis, Fusarium spp. (e.g., Fusarium culmorum, Fusarium oxysporium, Fusarium solani, Fusarium graminearum and Fusarium moniliforme), Gaeumannomyces graminis, Giberella fujikuroi, Giberella zeae, Helminthosporium graminearum, Monographella nivalis, Puccinia spp., Pyrenophora spp. (e.g., Pyrenophora graminea), Peronosclerospora spp., Peronspora spp., Phakopsora pachyrhizi, Phythium spp., Phoma spp., Phomopsis spp., Rhizoctonia solani, Septoria spp., Pseudocercosporella spp., Thelaviopsis spp., Tilletia spp., Rhizopus spp., Typhula spp., Ustilago spp., Sphacelotheca spp. (e.g., Spacelotheca reilliani), Thanatephorus cucumeris, and Verticillium spp.

The combinations of the present invention are particularly effective against fungal pathogens of the genus Fusarium, Pythium and/or Rhizoctonia.

The combinations of the invention can be formulated for a particular use. Preferably, the combination is formulated for protecting cultivated plants or their propagation materials. Advantageously, the combinations are formulated for seed treatment applications for controlling or preventing damage by nematodes, which are found in agriculture and forestry, and can particularly damage the plant in the early stages of its development.

Further, the combinations of the invention can be applied to the plant and/or part of the plant, in a conventional manner, such as foliar spray. Thus the benefit from the invention can be achieved either by (a) treating plant propagation material with a combination or (b) applying to the plant and/or part of the plant developing from the plant propagation material a combination, or both (a) and (b).

Further, the present invention also envisages soil application of the combinations of the invention to control the soil-dwelling nematodes and/or soil-borne pathogens. Methods of applying to the soil can be via any suitable method, which ensures that the combination penetrates the soil, for example, nursery tray application, in furrow application, soil drenching, soil injection, drip irrigation, application through sprinklers or central pivot, incorporation into soil (broad cast or in band) are such methods. Thus the benefit from the invention can be achieved either by (A) treating plant propagation material with a combination or (B) applying to the locus where control is desired, generally the planting site, a combination, or both (A) and (B). For example, a combination of the invention can be applied to bare soil, that is in the absence of plant or plant propagation material, and the plant propagation material can then advantageously be planted into such treated soil. Alternatively, the combination of the invention can be applied to the soil at the same time as the plant propagation material is planted into said soil, for example by sprinkling in granules of the composition. Another possibility is that the plant propagation material is planted into untreated soil and the combination of the invention is then applied to the locus of the planted plant propagation material, for example by drenching with a solution of the composition. Likewise, the benefit from the invention can be achieved either by (A) applying to the locus where control is desired, generally the planting site, a combination or (B) applying to the plant or parts of the plant developing from the plant propagation material a combination, or both (A) and (B).

The term “plant propagation material” is understood to denote all the generative parts of the plant, such as seeds, which can be used for the multiplication of the latter and vegetative plant material, such as cuttings and tubers (for example, potatoes). Accordingly, as used herein, part of a plant includes propagation material. There may be mentioned, e.g., the seeds (in the strict sense), roots, fruits, tubers, bulbs, rhizomes, parts of plants. Germinated plants and young plants, which are to be transplanted after germination or after emergence from the soil, may also be mentioned. These young plants may be protected before transplantation by a total or partial treatment by immersion.

Parts of plant and plant organs that grow at later point in time are any sections of a plant that develop from a plant propagation material, such as a seed. Parts of plant, plant organs, and plants can also benefit from the nematode and/or pathogenic damage protection achieved by the application of the combination on to the plant propagation material. In an embodiment, certain parts of plant and certain plant organs that grow at later point in time can also be considered as plant propagation material, which can themselves be applied (or treated) with the combination; and consequently, the plant, further parts of the plant and further plant organs that develop from the treated parts of plant and treated plant organs can also benefit from the nematode and/or pathogenic damage protection achieved by the application of the combination on to the certain parts of plant and certain plant organs.

Methods for applying or treating pesticidal active ingredients and mixtures thereof on to plant propagation material, especially seeds, are known in the art, and include dressing, coating, pelleting and soaking application methods of the propagation material.

The active ingredients can be applied to the seeds using conventional treating techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art.

In a preferred embodiment, the combination is applied or treated on to the plant propagation material by a method such that the germination is not induced; generally seed soaking induces germination because the moisture content of the resulting seed is too high. Accordingly, preferred examples of suitable methods for applying (or treating) a plant propagation material, such as a seed, is seed dressing, seed coating or seed pelleting and alike.

It is preferred that the plant propagation material is a seed. Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed either before or after the treatment or the treatment may be applied during the priming process.

Even distribution of the active ingredients and adherence thereof to the seeds is desired during propagation material treatment. Treatment could vary from a thin film (dressing) of the formulation containing the active ingredient(s) on a plant propagation material, such as a seed, where the original size and/or shape are recognizable to an intermediary state (such as a coating) and then to a thicker film (such as pelleting with many layers of different materials (such as carriers, for example, clays; different formulations, such as of other active ingredients; polymers; and colourants) where the original shape and/or size of the seed is no longer recognisable.

The seed treatment occurs to an unsown seed, and the term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil but would include any application practice that would target the seed during the planting process.

Preferably, the treatment occurs before sowing of the seed so that the sown seed has been pre-treated with the combination. In particular, seed coating or seed pelleting are preferred in the treatment of the combinations according to the invention. As a result of the treatment, the active ingredients in the combination are adhered on to the seed and therefore available for nematode control.

The treated seeds can be stored, handled, sowed and tilled in the same manner as any other active ingredient treated seed.

The combination according to the present invention is suitable for plants of the crops: cereals (wheat, barley, rye, oats, maize, rice, sorghum, triticale and related crops); beet (sugar beet and fodder beet); leguminous plants (beans, lentils, peas, soybeans); oil plants (rape, mustard, canola, sunflowers); cucumber plants (marrows, cucumbers, melons); fibre plants (cotton, flax, hemp, jute); vegetables (spinach, lettuce, asparagus, cabbages, broccoli, cauliflower, carrots, onions, tomatoes, peppers, potatoes, paprika); as well as ornamentals (flowers, shrubs, broad-leaved trees and evergreens, such as conifers). Especially suitable are cotton, maize, soybeans, sugar beet, cucumber plants and vegetables. Most suitable are soybeans.

Suitable target crops also include transgenic crop plants of the foregoing types. The transgenic crop plants used according to the invention are plants, or propagation material thereof, which are transformed by means of recombinant DNA technology in such a way that they are—for instance—capable of synthesizing selectively acting toxins as are known, for example, from toxin-producing invertebrates, especially of the phylum Arthropoda, as can be obtained from Bacillus thuringiensis strains; or as are known from plants, such as lectins; or in the alternative capable of expressing a herbicidal or fungicidal resistance. Examples of such toxins, or transgenic plants which are capable of synthesizing such toxins, have been disclosed, for example, in EP-A-0 374 753, WO 93/07278, WO 95/34656, EP-A-0 427 529 and EP-A-451 878 and are incorporated by reference in the present application.

The plant propagation material treated by a combination of the present invention are, therefore, resistant to nematode and/or pathogenic damage; accordingly, the present invention also provides nematode resistant plant propagation material which is treated with the combination and consequently at least, the active ingredients thereof are adhered on the propagation material, such a seed.

The seed treatment combination and composition can also comprise or may be applied together and/or sequentially with further active compounds. These further compounds can be other pesticidal active ingredients, fertilizers, antioxidants or micronutrient donors or other preparations that influence plant growth, such as inoculants.

A single pesticidal active ingredient may have activity in more than area of disease or pest control, for example, a pesticide may have fungicide, insecticide and nematicide activity. Specifically, aldicarb is known for insecticide, acaricide and nematicide activity, while metam is known for insecticide, herbicide, fungicide and nematicide activity, thiabendazole and captan can provide nematicide and fungicide activity, and thiodicarb is known for insecticide and molluscicide activity.

The combination of the present invention may be mixed with other pesticides, such as fungicides and insecticides.

Suitable examples of fungicides include carbamate fungicides (e.g., thiram), carboxamide fungicides (e.g., carboxin), phenylamide fungicides (e.g., metalaxyl, mefenoxam), phenylpyrrole fungicides (e.g., fludioxonil), strobilurin fungicides (e.g., azoxystrobin, fluoxastrobin, trifloxystrobin), triazole fungicides (e.g., myclobutanil, ipconazole, triadimenol), and mixtures thereof.

Suitable examples of insecticides include neonicotinoid insecticides (e.g., clothianidin, imidacloprid, thiamethoxam), pyrethroid insecticides (e.g., lambda-cyhalothrin, beta-cyfluthrin), benzoylurea insecticides (e.g., triflumuron), and mixtures thereof.

In a preferred embodiment, the combination further comprises one or more of (a) mefenoxam, (b) fludioxonil, (c) azoxystrobin, (d) clothianidin, (e) imidacloprid and/or (f) thiamethoxam.

In another preferred embodiment, the combination further comprises one or more of (a) metalaxyl, (b) trifloxystrobin, (c) ipconazole, (d) triadimenol, and/or (e) triflumuron.

In the event a combination of the invention also includes an insecticide then the pesticide spectrum of the combination is broadened to include pest control, such as control of pests selected from Insecta and Arachnida. In that instance, the combination can also be applied on the pest to control or prevent pest damage and protect the desired material (e.g., plant and parts of plant) from pest damage. Examples of pests include: from the order Lepidoptera (e.g., Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia spp., Cryptophlebia leucotreta, Crysodeixis includens, Cydia spp., Diatraea spp., Diparopsis castanea, Earias spp., Elasmopalpus spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiella, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichopiusia spp. and Yponomeuta spp.);

from the order Coleoptera (e.g., Agriotes spp., Anthonomus spp., Atomaria linearis, Ceutorhynchus spp., Chaetocnema tibialis, Cosmopolites spp., Curculio spp., Dermestes spp., Diabrotica spp., Epilachna spp., Eremnus spp., Gonocephalum spp., Heteronychus spp., Leptinotarsa decemlineata, Lissorhoptrus spp., Melolontha spp., Orycaephilus spp., Otiorhynchus spp., Phlyctinus spp., Phyllotreta spp., Popillia spp., Protostrophus spp., Psylliodes spp., Rhizopertha spp., Scarabeidae, Sitophilus spp., Sitotroga spp., Tenebrio spp., Tribolium spp. and Trogoderma spp.);
from the order Orthoptera (e.g., Blatta spp., Blattella spp., Gryllotalpa spp., Leucophaea maderae, Locusta spp., Periplaneta spp. and Schistocerca spp.);
from the order Isoptera (e.g., Reticulitermes spp.);
from the order Psocoptera (e.g., Liposcelis spp.);
from the order Anoplura (e.g., Haematopinus spp., Linognathus spp., Pediculus spp., Pemphigus spp. and Phylloxera spp.);
from the order Mallophaga (e.g., Damalinea spp. and Trichodectes spp.);
from the order Thysanoptera (e.g., Frankliniella spp., Hercinothrips spp., Taeniothrips spp., Thrips palmi, Thrips tabaci and Scirtothrips aurantii);
from the order Heteroptera (e.g., Dichelops melacanthus, Distantiella theobroma, Dysdercus spp., Euchistus spp., Eurygaster spp., Leptocorisa spp., Nezara spp., Piesma spp., Rhodnius spp., Sahlbergella singularis, Scotinophara spp. and Triatoma spp.);
from the order Homoptera (e.g., Aleurothrixus floccosus, Aleyrodes brassicae, Aonidiella spp., Aphididae spp., Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Paratoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri);
from the order Hymenoptera (e.g., Acromyrmex spp., Athalia rosae, Atta spp., Cephus spp., Diprion spp., Diprionidae spp., Gilpinia polytoma, Hoplocampa spp., Lasius spp., Monomorium pharaonis, Neodiprion spp., Solenopsis spp. and Vespa spp.);
from the order Diptera (e.g., Antherigona soccata, Bibio hortulanus, Ceratitis spp., Chrysomyia spp., Culex spp., Cuterebra spp., Dacus spp., Delia spp., Drosophila melanogaster, Liriomyza spp., Melanagromyza spp., Orseolia spp., Oscinella frit, Pegomyia hyoscyami, Phorbia spp., Rhagoletis pomonella, Sciara spp.); and
from the order Acarina (e.g., Acarus siro, Aceria sheldoni, Aculus schlechtendali, Amblyomma spp., Argas spp., Brevipalpus spp., Bryobia praetiosa, Calipitrimerus spp., Chorioptes spp., Dermanyssus gallinae, Eotetranychus carpini, Eriophyes spp., Hyalomma spp., Olygonychus pratensis, Ornithodoros spp., Panonychus spp., Phyllocoptruta oleivora, Polyphagotarsonemus latus, Psoroptes spp., Rhipicephalus spp., Rhizoglyphus spp., Sarcoptes spp., Tarsonemus spp. and Tetranychus spp.).

The weight ratio of active ingredients in the combination vary, for example, according to type of use, type of crop, the specific active ingredients in the combination, type of plant propagation material (if appropriate), but is such that the active ingredients in the combination is an effective proportion to provide the desired enhanced action (such as nematode and/or pathogenic control) and can be determined by trials known to one of ordinary skill in the art.

The weight ratio of active ingredient compounds is selected as to give the desired, for example synergistic, action. In general, the weight ratio would vary depending on the specific active ingredient and how many active ingredients are present in the combination. Generally, in the event the combination consists of two components (I) and (II) the weight ratio between components (I) and (II) is from 10000:1 to 1:1000, more preferably from 1000:1 to 1:100, most preferably from 100:1 to 1:10.

The rates of application (use) of the combination vary, for example, according to type of use, type of crop, the specific active ingredients in the combination, type of plant propagation material (if appropriate), but is such that the active ingredients in the combination is an effective amount to provide the desired enhanced action (such as nematode and/or pathogenic control) and can be determined by trials known to one of ordinary skill in the art.

Generally for seed treatment, application rates can vary from 0.1 μg to 10 mg of active ingredients per seed. Examples of application rates for seed treatment tend to be 0.01 mg-10 mg, preferably 0.1 mg-1.0 mg of component (I) per seed; and 0.1 μg-10 mg, and preferably 1.0 μg-1.0 mg of component (II) per seed.

In the event the combination comprises (a) abamectin as component (I) and acibenzolar-S-methyl as component (II), typical application rates for seed treatment, in particular on soybean, is 0.1-0.5 mg of abamectin per seed, and 2.5-10 g of acibenzolar-S-methyl per 100 kg seed.

In the event the combination comprises (a) abamectin as component (I) and acibenzolar-S-methyl as component (II), typical application rates for seed treatment, in particular on sugar beet, is 0.1-0.5 mg of abamectin per seed, and 2.5-10 g of acibenzolar-S-methyl per 100 kg seed.

In the event the combination comprises (a) abamectin as component (I) and acibenzolar-S-methyl as component (II), typical application rates for seed treatment, in particular on tomato, is 0.1-0.6 mg of abamectin per seed, and 1.0-500 μg of acibenzolar-S-methyl per seed.

In the event the combination comprises (a) abamectin as component (I) and acibenzolar-S-methyl as component (II), typical application rates for seed treatment, in particular on cucumber, is 0.1-0.6 mg of abamectin per seed, and 1.0-500 μg of acibenzolar-S-methyl per seed.

In the event the combination comprises (a) abamectin as component (I) and acibenzolar-S-methyl as component (II), typical application rates for seed treatment, in particular on cotton, is 0.1-0.3 mg of abamectin per seed, and 1.0-100 g of acibenzolar-S-methyl per 100 kg seed.

In the event the combination comprises (a) abamectin as component (I) and acibenzolar-S-methyl as component (II), typical application rates for seed treatment, in particular on maize, is 0.1-0.5 mg of abamectin per seed, and 1.0-100 g of acibenzolar-S-methyl per 100 kg seed.

In the event the combination comprises (g) a mixture of abamectin and thiabendazole as component (I) and acibenzolar-S-methyl as component (II), typical application rates for seed treatment, in particular on soybean, is 0.1-0.5 mg of abamectin per seed, 1.0-50 g of thiabendazole per 100 kg seed, and 2.5-10 g of acibenzolar-S-methyl per 100 kg seed. A most preferred embodiment is a combination wherein the application rate for seed treatment, in particular on soybean, is 0.15 mg of abamectin per seed, 20 g of thiabendazole per 100 kg seed, and 10.0 g of acibenzolar-S-methyl per 100 kg seed.

In the event the combination comprises (g) a mixture of abamectin and thiabendazole as component (I) and acibenzolar-S-methyl as component (II), typical application rates for seed treatment, in particular on sugar beet, is 0.1-0.5 mg of abamectin per seed, 1.0-50 g of thiabendazole per 100 kg seed, and 2.5-10 g of acibenzolar-S-methyl per 100 kg seed.

In the event the combination comprises (g) a mixture of abamectin and thiabendazole as component (I) and acibenzolar-S-methyl as component (II), typical application rates for seed treatment, in particular on tomato, is 0.1-0.6 mg of abamectin per seed, 1.0-50 g of thiabendazole per 100 kg seed, and 1.0-500 μg of acibenzolar-S-methyl per seed.

In the event the combination comprises (g) a mixture of abamectin and thiabendazole as component (I) and acibenzolar-S-methyl as component (II), typical application rates for seed treatment, in particular on cucumber, is 0.1-0.6 mg of abamectin per seed, 1.0-50 g of thiabendazole per 100 kg seed, and 1.0-500 μg of acibenzolar-S-methyl per seed.

In the event the combination comprises (g) a mixture of abamectin and thiabendazole as component (I) and acibenzolar-S-methyl as component (II), typical application rates for seed treatment, in particular on cotton, is 0.1-0.3 mg of abamectin per seed, 1.0-50 g of thiabendazole per 100 kg seed, and 1.0-100 g of acibenzolar-S-methyl per 100 kg seed.

In the event the combination comprises (g) a mixture of abamectin and thiabendazole as component (I) and acibenzolar-S-methyl as component (II), typical application rates for seed treatment, in particular on maize, is 0.1-0.5 mg of abamectin per seed, 1.0-50 g of thiabendazole per 100 kg seed, and 1.0-100 g of acibenzolar-S-methyl per 100 kg seed.

The components (I) or (II), and any other additional pesticides, may be used either in pure form, i.e., as a solid active ingredient, for example, in a specific particle size, or preferably together with at least one of the auxiliaries (also known as adjuvants) customary in formulation technology, such as extenders, e.g., solvents or solid carriers, or surface-active compounds (surfactants), in the form of a formulation, in the present invention. Generally, the components (I) and (II) are in the form of a formulation composition with one or more of customary formulation auxiliaries.

Therefore, the combination of components (I) and (II) are normally used in the form of formulations. The components (I) and (II) can be applied to the locus where control is desired either simultaneously or in succession at short interval, for example on the same day, if desired together with further carriers, surfactants or other application-promoting adjuvants customarily employed in formulation technology. In a preferred embodiment, (I) and (II) are applied simultaneously.

In the event components (I) and (II) are applied simultaneously in the present invention, they may be applied as a composition containing (1) and (11), in which case each of (I) and (II) can be obtained from a separate formulation source and mixed together (known as a tank-mix, ready-to-apply, spray broth, or slurry), optionally with other pesticides, or (I) and (II) can be obtained as single formulation mixture source (known as a pre-mix, concentrate, formulated compound (or product)), and optionally mixed together with other pesticides.

In an embodiment, the combination of the present invention is applied as a composition. Accordingly, the present invention is a composition comprising, as active ingredients, (I) and (II), and optionally other pesticides, and optionally one or more customary formulation auxiliaries; which may be in the form of a tank-mix or pre-mix composition.

In a preferred embodiment, each of the combinations of the invention are provided in the form of a pre-mix composition (or mixture): suitable examples being combinations of

(a) abamectin as component (I) and acibenzolar-S-methyl as component (II);
(b) aldicarb as component (I) and acibenzolar-S-methyl as component (II);
(c) oxamyl as component (I) and acibenzolar-S-methyl as component (II);
(d) thiodicarb as component (I) and acibenzolar-S-methyl as component (II);
(g) a mixture of abamectin and thiabendazole as component (I) and acibenzolar-S-methyl as component (II); and
(h′) a mixture of abamectin and thiophanate-methyl.

Examples of foliar formulation types for pre-mix compositions are:

GR: Granules (mainly used for direct soil treatment)
WP: wettable powders
WG: water dispersible granules (powders)
SG: water soluble granules
SL: soluble concentrates
EC: emulsifiable concentrate
EW: emulsions, oil in water
ME: micro-emulsion
SC: aqueous suspension concentrate
CS: aqueous capsule suspension
OD: oil-based suspension concentrate, and
SE: aqueous suspo-emulsion.

Whereas, examples of seed treatment formulation types for pre-mix compositions are:

WS: wettable powders for seed treatment slurry
LS: solution for seed treatment
ES: emulsions for seed treatment
FS: suspension concentrate for seed treatment
WG: water dispersible granules, and
CS: aqueous capsule suspension.

Examples of formulation types suitable for tank-mix compositions are solutions, dilute emulsions, suspensions, or a mixture thereof, and dusts.

As with the nature of the formulations, the methods of application, such as foliar, drench, spraying, atomizing, dusting, scattering, coating or pouring, are chosen in accordance with the intended objectives and the prevailing circumstances.

The tank-mix compositions are generally prepared by diluting with a solvent (for example, water) the one or more pre-mix compositions containing different pesticides, and optionally further auxiliaries.

Suitable carriers and adjuvants can be solid or liquid and are the substances ordinarily employed in formulation technology, e.g. natural or regenerated mineral substances, solvents, dispersants, wetting agents, tackifiers, thickeners, binders or fertilizers.

The formulations are prepared in known manner, e.g., by homogeneously mixing and/or grinding the active ingredients with extenders, e.g., solvents, solid carriers and, where appropriate, surface-active compounds (surfactants).

Suitable solvents include: aromatic hydrocarbons, preferably the fractions containing 8 to 12 carbon atoms, e.g. xylene mixtures or substituted naphthalenes, phthalates, such as dibutyl phthalate or dioctyl phthalate, aliphatic hydrocarbons, such as cyclohexane or paraffins, alcohols and glycols and their ethers and esters, such as ethanol, ethylene glycol, ethylene glycol monomethyl or monoethyl ether, ketones, such as cyclohexanone, strongly polar solvents, such as N-methyl-2-pyrrolidone, dimethyl sulfoxide or dimethylformamide, as well as vegetable oils or epoxidised vegetable oils, such as epoxidised coconut oil or soybean oil; or water.

The solid carriers used, e.g., for dusts and dispersible powders, are normally natural mineral fillers, such as calcite, talcum, kaolin, montmorillonite or attapulgite. In order to improve the physical properties it is also possible to add highly dispersed silicic acid or highly dispersed absorbent polymers. Suitable granulated adsorptive carriers include porous types, for example pumice, broken brick, sepiolite or bentonite, and suitable nonsorbent carriers are, for example, calcite or sand. In addition, a great number of pregranulated materials of inorganic or organic nature can be used, e.g., especially dolomite or pulverized plant residues.

Depending upon the nature of the active ingredient compounds to be formulated, suitable surface-active compounds are non-ionic, cationic and/or anionic surfactants having good emulsifying, dispersing and wetting properties. The term “surfactants” will also be understood as comprising mixtures of surfactants.

Particularly advantageous application-promoting adjuvants are also natural or synthetic phospholipids of the cephalin and lecithin series, e.g., phosphatidylethanolamine, phosphatidylserine, phosphatidylglycerol and lysolecithin.

Generally, a tank-mix formulation for foliar or soil application comprises 0.1 to 20%, especially 0.1 to 15%, active ingredient compounds, and 99.9 to 80%, especially 99.9 to 85%, of a solid or liquid auxiliaries (including, for example, a solvent such as water), where the auxiliaries can be a surfactant in an amount of 0 to 20%, especially 0.1 to 15%, based on the tank-mix formulation.

Typically, a pre-mix formulation for foliar application comprises 0.1 to 99.9%, especially 1 to 95%, active ingredient compounds, and 99.9 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Normally, a tank-mix formulation for seed treatment application comprises 0.25 to 80%, especially 1 to 75%, active ingredient compounds, and 99.75 to 20%, especially 99 to 25%, of a solid or liquid auxiliaries (including, for example, a solvent such as water), where the auxiliaries can be a surfactant in an amount of 0 to 40%, especially 0.5 to 30%, based on the tank-mix formulation.

Typically, a pre-mix formulation for seed treatment application comprises 0.5 to 99.9%, especially 1 to 95%, active ingredient compounds, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Whereas commercial products will preferably be formulated as concentrates (e.g., pre-mix composition (formulation)), the end user will normally employ dilute formulations (e.g., tank mix composition).

Preferred seed treatment pre-mix formulations are aqueous suspension concentrates. The formulation can be applied to the seeds using conventional treating techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art.

The Examples which follow serve to illustrate the formulations suitable for components (I) and (II), “active ingredient” denoting a combination of component (I) and component (II) in a specific mixing ratio.

### Formulation Examples

| Wettable powders | a) | b) | c) |
|---|---|---|---|
| active ingredient [I:II = 1:6(a), | 25% | 50% | 75% |
| 1:2(b), 1:1(c)] | | | |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders which can be diluted with water to give suspensions of the desired concentration.

| Dusts | a) | b) | c) |
|---|---|---|---|
| active ingredient [I:II = 1:6(a), | 5% | 6% | 4% |
| 1:2(b), 1:10(c)] | | | |
| Talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can be used for dry dressings for seed.

| Suspension concentrates | (a) | (b) |
|---|---|---|
| active ingredient (I:II = 1:1(a); 1:8(b)) | 5% | 30% |
| propylene glycol | 10% | 10% |
| Tristyrylphenol ethoxylates | 5% | 6% |
| sodium lignosulfonate | — | 10% |
| carboxymethylcellulose | — | 1% |
| silicone oil (in the form of a 75% emulsion in water) | 1% | 1% |
| Colour pigment | 5% | 5% |
| water | 74% | 37% |

The finely ground active ingredient is intimately mixed with the adjuvants, giving a suspension concentrate from which suspensions of any desired dilution can be obtained by dilution with water. Alternatively, a suspension of the active ingredients and auxiliaries (including water) is wet milled with a bead-mill to achieve a stable formulation and with the appropriate treatment characteristics.

Using such dilutions plant propagation material can be treated and protected against damage, for example, from pathogen(s), by spraying, pouring or immersing.

The active ingredient combinations according to the invention are distinguished by the fact that they are especially well tolerated by plants and are environmentally friendly.

Each active ingredient combination according to the invention is especially advantageous for the treatment of plant propagation material.

In a preferred embodiment, each of the combinations of the present invention is a plant propagation material, preferably seed, treating composition.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The invention is illustrated by the following, non-limiting, examples.

### Biological Examples:

The expected percentage of disease control to be expected, E, for a given combination of two active ingredients can be calculated as follows (see COLBY, S. R. “Calculating synergistic and antagonistic response of herbicide combinations”. Weeds 15, pages 20-22; 1967):

E=X+Y−(X*Y)/100

wherein:
X=% disease control in the case of treatment with active ingredient (A) at a defined application rate, in comparison with untreated control (=0%).
Y=% disease control in the case of treatment with active ingredient (B) at a defined application rate, in comparison with untreated control.
E=expected percentage of disease control (% disease control in comparison with untreated control) after treatment with active ingredient (A) and active ingredient (B) at a defined application rate.
If the observed percentage of disease control O is greater than the expected value E, there is a synergistic effect.

### Trial Description:

### 1. Cultivation of the Soybean Plants

Soybeans were planted into “cone-tainers” (3.8 cm cell diameter; 14 cm cell depth; the Ray Leach “Cone-tainer” Single Cell System consists of individual cells that can be arranged within a tray and is available from Stuewe and Sons Incorporated) in 70% sand 30% clay loam soil mix (one soybean seed per cone-tainer). The cone-tainers contained a 10 cm by 10 cm piece of cheesecloth at the bottom to prevent the soil coming out of the bottom. The soil mix was autoclaved for 60 minutes prior to use. Extra cone-tainers were planted which allowed similar size soybean plants to be selected and placed into a randomized complete block design with 3 replications of each treatment. The cone-tainers were placed into a growth chamber set at 27° C. and 14 hour day light periods. The air temperature in the growth chamber was monitored in 15 minutes intervals using a watchdog data collector. The cone-tainers were watered daily as needed to ensure that overwatering did not occur. The water source was supplemented with fertilizer to promote plant growth.

### 2. Preparation of the Soybean Cyst Nematode (Heterodera Glycines) Egg Inoculant

Soil was collected from a field with “HG type” 2.5.7 (The “HG type” test is a greenhouse test that provides information on how well a soybean cyst nematode “Heterodera glycines, HG” population can reproduce in a particular soil). The soil was added to water and poured over a 25 mesh sieve over a 60 mesh sieve to collect the cysts. The soil solution collected on the 60 mesh sieve was then placed onto a 120 mesh sieve over 500 mesh sieve. The cysts were broken open to release the eggs by taking a rubber stopper and rubbing the soil solution on the 120 mesh sieve. The egg solution was then centrifuged in a sugar concentration of 453.6 grams per liter of water at 2100 rpm for two minutes to remove soil debris. The clean egg solution was then concentrated to achieve 6,000 eggs per mL of solution.

### 3. Inoculation and Cultivation of the Soybean Plants

The soybean plants were inoculated with 6,000 soybean cyst nematode eggs 7 days after the planting of the soybeans into the cone-tainers. The plants were inoculated by placing a pencil tip 2.5 cm into the soil next to the seedling. One mL of egg solution was pipetted into the opening. Water from a squirt bottle was used to force the soil to collapse into the inoculation site, this was used to prevent soil compaction and air pockets to form in the soil. The inoculated plants were placed into a growth chamber set at 27° C. and 14 hour day light periods. The air temperature in the growth chamber was monitored in 15 minutes intervals using a watchdog data collector. The plants were watered daily as needed to ensure that overwatering did not occur and drown the nematodes. The water source was supplemented with fertilizer to promote plant growth.

4. Determination of the Number of Soybean Cyst Nematode Eggs 30 Days after Inoculation

The plants were removed from the growth chamber 30 days after inoculation to assess the nematode reproduction factor. The soil and roots of each cone-tainer were washed to extract the cysts. A similar procedure as described under 2. was followed to isolate the nematode eggs. The total number of soybean cyst nematode eggs present in each sample was determined. The average values from the 3 replicates for each treatment were used in the calculations below.

| | |
|---|---|
| (A-a) | abamectin |
| (A-g) | a mixture of abamectin and thiabendazole |
| (B) | acibenzolar-S-methyl |

TABLE 1
Observed Rf values at 30 days after inoculation

| Treatment | Rate | Rf |
|---|---|---|
| untreated control | — | 6.6 |
| (A-a) | 0.15 mg abamectin/seed | 2.5 |
| (A-g) | 0.15 mg abamectin/seed + | 3.7 |
| | 20 g thiabendazole/100 kg seed | |
| (B-1) | 2.5 g acibenzolar-S-methyl/100 kg seed | 4.1 |
| (B-2) | 5.0 g acibenzolar-S-methyl/100 kg seed | 2.7 |
| (B-3) | 10.0 g acibenzolar-S-methyl/100 kg seed | 3.9 |
| (A-a) + (B-1) | 0.15 mg abamectin/seed + | 1.8 |
| | 2.5 g acibenzolar-S-methyl/100 kg seed | |
| (A-a) + (B-2) | 0.15 mg abamectin/seed + | 4.3 |
| | 5.0 g acibenzolar-S-methyl/100 kg seed | |
| (A-a) + (B-3) | 0.15 mg abamectin/seed + | 2.7 |
| | 10.0 g acibenzolar-S-methyl/100 kg seed | |
| (A-g) + (B-1) | 0.15 mg abamectin/seed + | 2.2 |
| | 20 g thiabendazole/100 kg seed + | |
| | 2.5 g acibenzolar-S-methyl/100 kg seed | |
| (A-g) + (B-2) | 0.15 mg abamectin/seed + | 1.8 |
| | 20 g thiabendazole/100 kg seed + | |
| | 5.0 g acibenzolar-S-methyl/100 kg seed | |
| (A-g) + (B-3) | 0.15 mg abamectin/seed + | 1.1 |
| | 20 g thiabendazole/100 kg seed + | |
| | 10.0 g acibenzolar-S-methyl/100 kg seed | |

The nematode reproduction factor Rf = (Pf + 1)/(Pi + 1)
wherein
Pf is the number of eggs 30 days after inoculation, and
Pi is the number eggs inoculated into each treatment.

TABLE 2
Expected percentage of disease control E

| Treatment | Rate | E |
|---|---|---|
| (A-a) + (B-1) | 0.15 mg abamectin/seed + | 76% |
| | 2.5 g acibenzolar-S-methyl/100 kg seed | |
| (A-a) + (B-2) | 0.15 mg abamectin/seed + | 85% |
| | 5.0 g acibenzolar-S-methyl/100 kg seed | |
| (A-a) + (B-3) | 0.15 mg abamectin/seed + | 78% |
| | 10.0 g acibenzolar-S-methyl/100 kg seed | |
| (A-g) + (B-1) | 0.15 mg abamectin/seed + | 65% |
| | 20 g thiabendazole/100 kg seed + | |
| | 2.5 g acibenzolar-S-methyl/100 kg seed | |
| (A-g) + (B-2) | 0.15 mg abamectin/seed + | 77% |
| | 20 g thiabendazole/100 kg seed | |
| | 5.0 g acibenzoiar-S-methyl/100 kg seed | |
| (A-g) + (B-3) | 0.15 mg abamectin/seed + | 67% |
| | 20 g thiabendazole/100 kg seed + | |
| | 10.0 g acibenzolar-S-methyl/100 kg seed | |

TABLE 3
Observed percentage of disease control O

| Treatment | Rate | O |
|---|---|---|
| (A-a) + (B-1) | 0.15 mg abamectin/seed + | 73% |
| | 2.5 g acibenzolar-S-methyl/100 kg seed | |
| (A-a) + (B-2) | 0.15 mg abamectin/seed + | 35% |
| | 5.0 g acibenzolar-S-methyl/100 kg seed | |
| (A-a) + (B-3) | 0.15 mg abamectin/seed + | 59% |
| | 10.0 g acibenzolar-S-methyl/100 kg seed | |
| (A-g) + (B-1) | 0.15 mg abamectin/seed + | 67% |
| | 20 g thiabendazole/100 kg seed + | |
| | 2.5 g acibenzolar-S-methyl/100 kg seed | |
| (A-g) + (B-2) | 0.15 mg abamectin/seed + | 73% |
| | 20 g thiabendazole/100 kg seed + | |
| | 5.0 g acibenzolar-S-methyl/100 kg seed | |
| (A-g) + (B-3) | 0.15 mg abamectin/seed + | 83% |
| | 20 g thiabendazole/100 kg seed + | |
| | 10.0 g acibenzolar-S-methyl/100 kg seed | |

1. A pesticidal combination comprising at least two active ingredient components optionally together with one or more customary formulation auxiliaries, wherein component (I) is one or more nematicide(s) and component (II) is one or more plant activator(s).

2. A combination according to claim 1 wherein
(a) abamectin is component (I) and acibenzolar-S-methyl is component (II);
(b) aldicarb is component (I) and acibenzolar-S-methyl is component (II);
(c) oxamyl is component (I) and acibenzolar-S-methyl is component (II); or
(d) thiodicarb is component (I) and acibenzolar-S-methyl is component (II).

3. A combination according to claim 1 wherein
(e) a mixture of abamectin and benomyl is component (I) and acibenzolar-S-methyl is component (II);
(f) a mixture of abamectin and captan is component (I) and acibenzolar-S-methyl is component (II);
(g) a mixture of abamectin and thiabendazole is component (I) and acibenzolar-S-methyl is component (II);
(h) a mixture of abamectin and thiophanate-methyl is component (I) and acibenzolar-S-methyl is component (II);
(j) a mixture of aldicarb and benomyl is component (I) and acibenzolar-S-methyl is component (II);
(k) a mixture of aldicarb and captan is component (I) and acibenzolar-S-methyl is component (II);
(m) a mixture of aldicarb and thiabendazole is component (I) and acibenzolar-S-methyl is component (II);
(n) a mixture of aldicarb and thiophanate-methyl is component (I) and acibenzolar-S-methyl is component (II);
(o) a mixture of oxamyl and benomyl is component (I) and acibenzolar-S-methyl is component (II);
(p) a mixture of oxamyl and captan is component (I) and acibenzolar-S-methyl is component (II);
(q) a mixture of oxamyl and thiabendazole is component (I) and acibenzolar-S-methyl is component (II);
(r) a mixture of oxamyl and thiophanate-methyl is component (I) and acibenzolar-S-methyl is component (II);
(s) a mixture of thiodicarb and benomyl is component (I) and acibenzolar-S-methyl is component (II);
(t) a mixture of thiodicarb and captan is component (I) and acibenzolar-S-methyl is component (II);
(u) a mixture of thiodicarb and thiabendazole is component (I) and acibenzolar-S-methyl is component (II); or
(v) a mixture of thiodicarb and thiophanate-methyl is component (I) and acibenzolar-S-methyl is component (II).

4. A combination according to claim 1 wherein the combination further comprises one or more fungicides.

5. A combination according to claim 1, wherein the combination further comprises one or more fungicides selected from the group comprising carbamate fungicides, carboxamide fungicides, phenylamide fungicides, phenylpyrrole fungicides, strobilurin fungicides and triazole fungicides, and mixtures thereof.

6. A combination according to claim 1, wherein the combination further comprises one or more fungicides selected from the group comprising thiram, carboxin, metalaxyl, mefenoxam, fludioxonil, azoxystrobin, fluoxastrobin, trifloxystrobin, myclobutanil, ipconazole and triadimenol.

7. A combination according to claim 1, wherein the combination further comprises mefenoxam.

8. A combination according to claim 1, wherein the combination further comprises fludioxonil.

9. A combination according to claim 1, wherein the combination further comprises azoxystrobin.

10. A combination according to claim 1, wherein the combination further comprises one or more insecticides.

11. A combination according to claim 1, wherein the combination further comprises one or more insecticides selected from the group comprising neonicotinoid insecticides, pyrethroid insecticides and benzoylurea insecticides, and mixtures thereof.

12. A combination according to claim 1, wherein the combination further comprises one or more insecticides selected from the group comprising clothianidin, imidacloprid, thiamethoxam, lambda-cyhalothrin, beta-cyfluthrin and triflumuron.

13. A combination according to claim 1, wherein the combination further comprises clothianidin.

14. A combination according to claim 1, wherein the combination further comprises imidacloprid.

15. A combination according to claim 1, wherein the combination further comprises thiamethoxam.

16. A combination according to claim 1, in the form of a plant propagation material treating pesticidal composition.

17. A method of controlling or preventing nematode and/or pathogenic damage in a plant propagation material, a plant, and/or plant organs that grow at a later point in time, which comprises applying on the plant, part of the plant, or the surrounding area thereof the combination, as defined in, claim 1, in any desired sequence or simultaneously.

18. A method of protecting a plant propagation material, a plant, and/or plant organs that grow at a later point in time against nematode and/or pathogenic damage, which comprises applying to the plant, part of the plant, or the surrounding area thereof the combination, as defined in claim 1, in any desired sequence or simultaneously.

19. A method of improving the growing characteristics of a plant, and/or plant organs that grow at a later point in time, which comprises applying to the plant, part of the plant, or the surrounding area thereof the combination, as defined in claim 1, in any desired sequence or simultaneously.

20. A method according to claim 17, wherein the active ingredient components of the combination, are applied simultaneously.

21. A method according to, claim 18, wherein the combination, is applied on plant propagation material.

22. A plant propagation material treated with the combination defined in claim 1.

23. A method according to, claim 17, wherein the nematode to be controlled or prevented is a root-knot nematode or a cyst-forming nematode.

24. A method according to claim 23, wherein a mixture of abamectin and thiabendazole is the active ingredient component (I) and acibenzolar-S-methyl is the active ingredient component (II).
